(19) United States
(12) Patent Application Publication
(10) Pub. No.: US 2008/0215078 A1
(43) Pub. Date: Sep. 4, 2008

(54) SURGICAL BLADE AND TROCAR SYSTEM

(76) Inventor: Michael D. Bennett, Honolulu, HI (US)

Correspondence Address: SHEPPARD, MULLIN, RICHTER & HAMPTON LLP, 333 SOUTH HOPE STREET, 48TH FLOOR, LOS ANGELES, CA, 90071-1448, US

(21) Appl. No.: 12/020,873

(22) Filed: Jan. 28, 2008

Related U.S. Application Data
(60) Provisional application No. 60/898,653, filed on Jan. 31, 2007.

(51) Int. Cl. A61F 9/007

(52) U.S. Cl. 606/166

(57) ABSTRACT

The present invention provides an improved surgical blade and trocar system for accessing the retina and other parts of the eye while doing vitreo-retinal and cataract surgeries, including surgeries for macular degeneration. The eye surgeon uses an improved surgical blade for vitreo-retinal and cataract surgeries having a generally flat, V-shaped, W-shaped, or “extended W” shaped cross-section. Using the improved surgical blade, the surgeon creates a multi-planar, self-sealing surgical wound, first by directing the surgical blade substantially perpendicular to the eye surface, then redirecting the blade to follow the general curvature of the eye globe, and finally redirecting the blade to enter the interior of the eye. The improved surgical blade is used with an improved trocar system having two main parts-a relatively rigid, wide-mouthed outer segment and a generally thin-walled, collapsible plastic polymer or metal mesh sleeve that spans the surgical wound and substantially molds to its contour. The improved surgical blade and trocar system can be adapted for use in either vitreo-retinal or cataract surgeries.

### CROSS-REFERENCE TO RELATED APPLICATION

Priority is claimed to U.S. Provisional Application Ser. No. 60/898,653, filed on Jan. 31, 2007, the contents of which are incorporated by reference in their entirety.

### FIELD OF THE INVENTION

The present invention relates generally to surgical blades and trocar systems for use in eye surgery and, more particularly, to a self-sealing, pressure-regulating surgical blade and trocar system for use in sutureless vitreo-retinal and cataract surgery.

### BACKGROUND OF THE INVENTION

Vitreo-retinal surgery (pars plana vitrectomy) is one of the fastest growing areas in ophthalmic surgery. With newer equipment and greater skill levels among surgeons, vitreo-retinal surgeries are being performed for an increasing number of conditions. But vitreo-retinal surgery still entails significant risks, and thus there is a need for safer and more efficient ways to perform such surgeries.

In performing vitreo-retinal surgery, surgeons have historically performed 20-gauge sclerotomies that provide for efficient vitreous removal and that allow the surgeons to use a wide variety of sturdy 20-gauge surgical instruments. To perform a 20-gauge sclerotomy, surgeons currently make a straight, single-pane, slit-like entry into the eye perpendicular to the eye wall. Current sclerotomy blades (MVR blades) are effective at making such an entry. The length of the blade point allows for rapid, full-thickness penetration through the sclera.

Unfortunately, current 20-gauge sclerotomies entail an undesirably large incision that requires sutures to close the wound. Without sutures, the 20-gauge wound cannot overcome the intraocular pressure and close on its own, leading to post-operative hypotony. Sutures increase the amount of time needed to complete the surgery, slow down visual recovery time, and boost the risk of infection, among other things. There is a need for improved surgical tools techniques that would allow surgeons to use the present 20-gauge instruments in a way that would also allow the surgical wound to heal without sutures.

Newer 23- and 25-gauge trocar procedures (collectively referred to as 25-gauge for simplicity) do offer a “self-sealing” option, whereby the surgical wound heals without sutures because of the wound's smaller size. Current 25-gauge trocar inserters use a rigid, needle-like entry device that creates a round, straight hole through the scleral wall. The outer segment of the trocar is generally cylindrical and pivots on the surface of the eye as the surgeon pivots the surgical instrument to move about the interior of the eye. The outer segment pivots with respect to the eye surface because the outer segment is rigidly attached to the trocar's rigid inner segment. Because 25-gauge trocar procedures can be self-sealing, inflammation is reduced and visual recovery is faster, as compared with current 20-gauge procedures.

Unfortunately, the current 25-gauge trocar procedures have serious shortcomings pertaining to, among other things, port-based flow limitations and the excessive flexibility of small 25-gauge instruments. Because 25-gauge instruments are so flexible, they easily bend within the trocar's rigid inner segment and move within the eye in ways that are confusing and counter-intuitive. Partly as a result, intra-ocular time during surgery is greater. Moreover, the outer segment of the trocar can harm the eye surface as it pivots. Thus, the newer 25-gauge trocar procedures are not a satisfactory solution to the problems posed by current 20-gauge sclerotomies.

Cataract surgery is likewise a fast growing area. But current cataract surgery also requires a large incision of such a size and nature that undesirable risks are posed to the patient. As with vitreo-retinal surgery, there is a need for safer and more efficient ways to perform cataract surgeries.

While 25-gauge trocar systems are currently needed to perform sutureless vitreo-retinal surgeries, non-trocar methods have been disclosed for performing sutureless cataract surgeries. For example, U.S. Pat. No. 6,171,324 to Cote et al. discloses a corneal marker and a method of using a corneal marker. The surgical method involves forming a multi-planar tunnel in the cornea, as shown in FIG. 9 of the patent. To create the multi-planar tunnel, the surgeon creates a groove in the corneal or limbal tissue to a depth of about 0.3 mm to about 0.6 mm. After forming the groove, the surgeon angles the surgical knife substantially parallel to the corneal surface and cuts a tunnel through the corneal tissue. After forming the tunnel, the surgeon angles the knife down, causing the blade to applanate the cornea. Because of the zigzag shape of the incision, intraocular pressure can close the tunnel, preventing leakage and removing the need for sutures.

Current trocar systems cannot be used with this zigzag incision, because current trocar systems use a rigid, needle-like entry device. After cutting the zigzag incision, the surgeon simply inserts the desired surgical instrument through the incision without the benefit of a trocar. Without a trocar, the surgical instrument can rub against the edges of the wound, causing a distortion or “rounding” of the wound and harming the surgical ocular surface. Thus, although the zigzag incision allows for a sutureless cataract surgery, it presently has shortcomings that would be desirable to avoid.

It should thus be appreciated that there exists a need for safer and more efficient ways to perform vitreo-retinal and cataract surgeries that overcome the drawbacks of current surgical tools and techniques, as described above. The present invention fulfills this need and provides further related advantages.

### SUMMARY OF THE INVENTION

In view of the foregoing, it is an object of the present invention to provide a safer and more efficient way to perform vitreo-retinal and cataract surgeries. The present invention generally provides an improved surgical blade and trocar system for accessing the retina and other parts of the eye while doing vitreo-retinal and cataract surgeries, including surgeries for macular degeneration.

One aspect of the present invention involves an improved surgical blade. In one embodiment, the present invention provides a new sclerotomy blade having relatively square shoulders allowing the surgeon to create a reproducible sclerotomy. Using the new blade, a surgeon can create a surgical wound that narrows in diameter from the scleral surface to the choroidal-sclera junction.

In another embodiment, the present invention provides new surgical blades for vitreo-retinal and cataract surgeries having a generally V- or modified W-shaped cross-section. By using a surgical blade having a generally V- or W-shaped cross-section, the surgeon can create an interlocking wound that will interdigitate or become interlocked like the fingers of folded hands. When stretched, the interlocking wound will permit a larger access opening while maintaining the shortest possible end-to-end measurement. The interlocking wound will generally seal stronger and be less likely to deform or open due to intra-ocular pressure, eyelid blinking, or hand rubbing. In a preferred embodiment, the surgical blade has a V- or W-shaped cross section, although other cross-sections permitting the creation of an interlocking wound are encompassed within the scope of the present invention, including surgical blades having an “extended W” shaped, arc-shaped, or U-shaped cross section. The scope of the present invention encompasses blade cross-sections that shorten the distance between the two ends of the surgical wound, while at the same time increasing the relative surface area of the wound.

Another aspect of the present invention involves the creation of a multi-planar, self-sealing surgical wound in vitreo-retinal surgeries. The wound is self-sealing due to the wound's architecture and trajectory, even when 20-gauge instruments are used. Because the wound is self-sealing, the patient can enjoy a speedier recovery. In one form, the wound narrows in diameter from the scleral surface to the choroidal-sclera junction.

To create the multi-planar wound, the surgeon directs the surgical blade substantially perpendicular to the scleral surface, creating a wound about 1.0 mm wide to a depth of about 0.25 mm in the sclera. Next, the surgeon redirects the blade to follow the general curvature of the eye globe. The surgeon then advances the blade, creating an approximately 0.75 to 1.0 mm tunnel. The surgeon then redirects the blade to create a full-thickness sclerotomy and entry into the eye.

A further aspect of the present invention involves an improved trocar having two main parts—a relatively rigid, wide-mouthed outer segment and a generally thin-walled, collapsible plastic or metal mesh sleeve that spans the surgical wound and substantially molds to its contour. The improved trocar can be adapted for use in either vitreo-retinal or cataract surgeries.

In one embodiment, the trocar has a relatively wide-mouthed (approximately 18+ gauge) opening and a generally funnel-shaped internal aspect, allowing for full rotation of surgical instruments and minimizing the bending of surgical instruments. The trocar also has a relatively large stability platform generally shaped to mate to the surface curvature of the eye globe. Additionally, the trocar glows in the dark, allowing a surgeon to locate the trocar easily if the operating room is dark. The trocar further has an external funnel shape allowing a surgeon to remove the trocar rapidly and easily at the conclusion of surgery.

In one embodiment, the trocar sleeve has generally thin walls that substantially mold to the shape of the surgical wound. The sleeve generally follows the wound and is held relatively securely in place. The sleeve is generally collapsible, effectively closing itself and minimizing the need for plugs when the surgeon removes a surgical instrument from the trocar. The sleeve is also relatively flexible, permitting increased mobility. The sleeve additionally provides predictability by minimizing the bending of surgical instruments. Furthermore, the sleeve is adaptive, allowing a surgeon to use any current size instrument (20, 23, 25 or smaller gauge).

Other features and advantages of the present invention should become apparent from the following description of the preferred embodiments, taken in conjunction with the accompanying drawings, which illustrate, by way of example, the principles of the invention.

### BRIEF DESCRIPTION OF THE DRAWINGS

Embodiments of the present invention will now be described, by way of example only, with reference to the following drawings, in which:

FIG. 1 is a side view of a preferred embodiment of a trocar system, in accordance with the principles of the present invention, showing a surgical instrument inserted into the trocar system;

FIG. 2 is a perspective view of a preferred embodiment of the outer segment of a trocar system, in accordance with the principles of the present invention;

FIG. 3 is a side view of a preferred embodiment of a straight surgical blade adapted for use in vitreo-retinal surgery, in accordance with the principles of the present invention;

FIG. 4a is a side view of a preferred embodiment of a V-shaped surgical blade, in accordance with the principles of the present invention;

FIG. 4b is a front view of a preferred embodiment of a V-shaped surgical blade, in accordance with the principles of the present invention;

FIG. 5a is a side view of a preferred embodiment of a W-shaped surgical blade, in accordance with the principles of the present invention;

FIG. 5b is a front view of a preferred embodiment of a W-shaped surgical blade, in accordance with the principles of the present invention;

FIG. 5c is a side view of a preferred embodiment of an “extended W” shaped surgical blade, in accordance with the principles of the present invention;

FIG. 5d is a front view of a preferred embodiment of an “extended W” shaped surgical blade, in accordance with the principles of the present invention;

FIG. 6 is a side cross-sectional view showing a surgical blade being directed substantially perpendicular to the eye surface, in accordance with the principles of the present invention;

FIG. 7 is a side cross-sectional view showing a surgical blade being directed to follow the general curvature of the eye globe, in accordance with the principles of the present invention;

FIG. 8 is a side cross-sectional view showing a surgical blade being directed to enter the interior of the eye, in accordance with the principles of the present invention;

FIG. 9 is a perspective cross-sectional view of a preferred embodiment of a trocar system, in accordance with the principles of the present invention, showing the trocar system inserted into an eyeball with a surgical instrument inserted into the trocar system;

FIG. 10 is a side cross-sectional view of a preferred embodiment of a trocar system, in accordance with the principles of the present invention, showing the trocar system inserted into an eyeball with a surgical instrument inserted into the trocar system;

FIG. 11 is a perspective cross-sectional view of a preferred embodiment of a trocar system, in accordance with the principles of the present invention, showing the trocar system inserted into an eyeball but without a surgical instrument inserted into the trocar system;

FIG. 12 is a side cross-sectional view of a preferred embodiment of a trocar system, in accordance with the principles of the present invention, showing the trocar system inserted into an eyeball but without a surgical instrument inserted into the trocar system;

### DETAILED DESCRIPTION OF THE PREFERRED EMBODIMENTS

The present invention is directed to safer and more efficient surgical tools and techniques to perform vitreo-retinal and cataract surgeries. The present invention generally provides an improved surgical blade and trocar system for accessing the retina and other pats of the eye while doing vitreo-retinal and cataract surgeries, including surgeries for macular degeneration.

In one embodiment, the present invention provides a pre-sterilized, disposable trocar system. The trocar system is meant for single use only and does not require assembly by the user. With minimal training, a vitreo-retinal or cataract specialist should adapt intuitively to this improved system. Based upon a concept of minimally invasive surgery, this trocar system can be used to create a self-sealing, multi-planar scleral or cataract incision using a new trocar device that improves both patient safety and surgical efficiency, as described further below.

### Trocar System

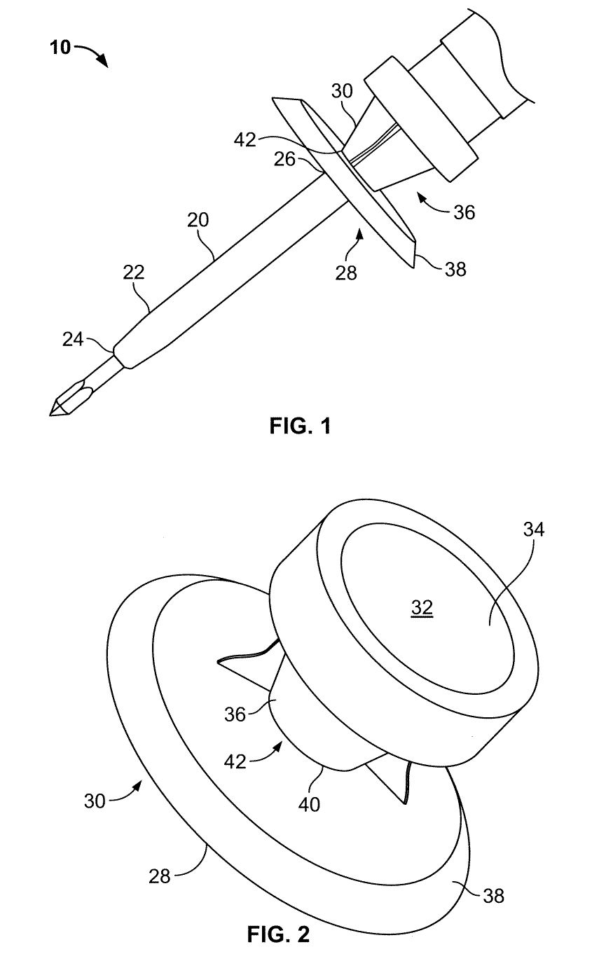

FIG. 1 shows a preferred embodiment of a trocar system 10, in accordance with the principles of the present invention. The trocar system 10 comprises two main parts—a generally thin-walled, collapsible, flexible plastic polymer, possibly fenestrated, or metal mesh sleeve 20 that spans the surgical wound and substantially molds to its contour and a relatively rigid, wide-mouthed outer segment 30 that glows or illuminates in the dark. The improved trocar system 10 can be adapted for use in either vitreo-retinal or cataract surgeries.

Sleeve 20 has generally thin walls 22 that substantially mold to the shape of the surgical wound. The sleeve 20 generally follows the wound and is held relatively securely in place. The sleeve 20 is generally collapsible, effectively closing itself when the surgeon removes a surgical instrument from the trocar system 10. The sleeve 20 is also relatively flexible, permitting increased mobility. The sleeve 20 additionally provides predictability by minimizing the bending of surgical instruments. Furthermore, the sleeve is adaptive, allowing a surgeon to use any current size instrument (20, 23, or 25 gauge). In one embodiment, the walls 22 of the sleeve 20 are comprised of a polymer shaped like a hose that is relatively rigid longitudinally (resists collapsing end-to-end) and is easily collapsible latitudinally. In another embodiment, the walls 22 are comprised of another polymer or metal mesh with similar characteristics. The scope of the present invention encompasses the walls 22 being comprised of other materials that accomplish the goals of the invention.

Sleeve 20 is generally shaped like a hollow cylinder, having a bottom end 24 that defines a 20-gauge opening and a top end 26 that also defines a 20-gauge opening. The top end 26 of sleeve 20 is connected to the bottom side 28 of the outer segment 30, although the scope of the present invention encompasses the top end 26 of sleeve 20 being connected to a different part of the outer segment 30.

### Outer Segment

FIG. 2 shows a preferred embodiment of the outer segment 30, in accordance with the principles of the present invention. The outer segment 30 has a relatively wide-mouthed (approximately 18+ gauge) opening 32 and a generally funnel-shaped internal aspect 34, allowing for full rotation of surgical instruments and minimizing the bending of surgical instruments. The outer segment 30 glows in the dark, allowing a surgeon to locate the outer segment 30 easily if the operating room is dark. The outer segment 30 also has a generally funnel-shaped external guide piece 36, allowing a surgeon to remove the trocar system 10 rapidly and easily at the conclusion of surgery.

The outer segment 30 additionally has a relatively large stability platform 38 generally shaped to mate to the surface curvature of the eye globe. The stability platform 38 is generally shaped like a flat doughnut, having an inner perimeter 40 that is connected to the bottom end 42 of the funnel-shaped external guide piece 36. The bottom side 28 of the stability platform 38 is generally concave shaped to contour to the eye curvature.

### Straight Surgical Blade

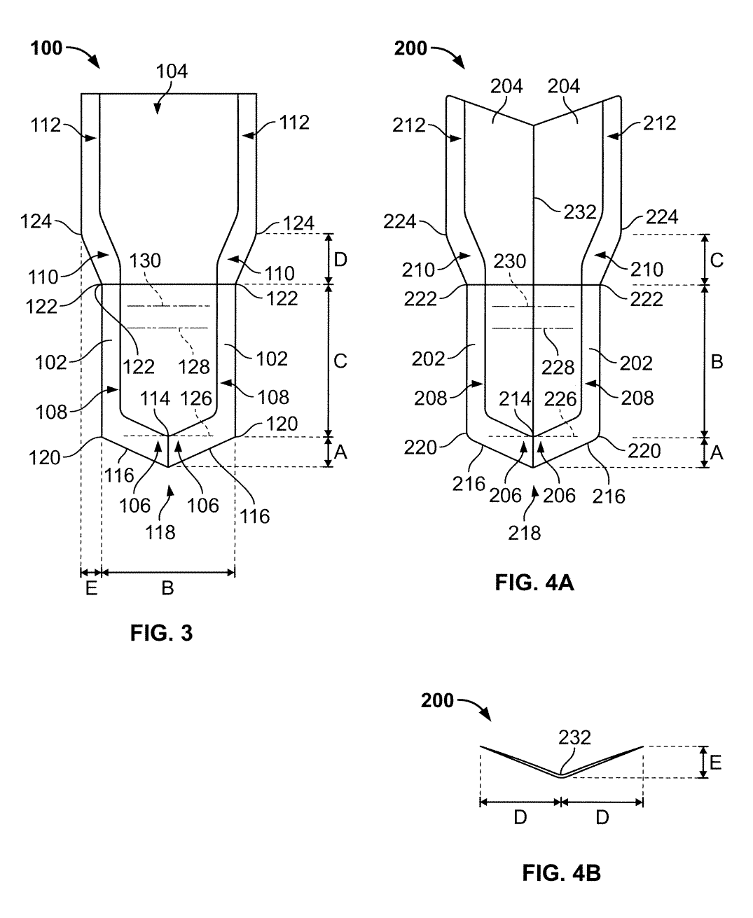

FIG. 3 shows a preferred embodiment of a straight surgical blade 100 adapted for use in vitreo-retinal surgery, in accordance with the principles of the present invention. The straight surgical blade 100 has two opposed cutting surfaces 102 that mirror each other and substantially surround a flat center portion 104. The two opposed cutting surfaces 102 are comprised of two forward cutting surfaces 106, two lower side cutting surfaces 108, two middle cutting surfaces 110, and two upper side cutting surfaces 112. The two forward cutting surfaces 106 meet seamlessly at the forward point 114 of the center portion 104.

Together, the two forward cutting surfaces 106 form a generally triangular-shaped forward end 116 of the straight surgical blade 100, having a forward point 118 and two lower apexes 120. At the forward point 118, the cutting surface is about 0.25 mm deep vertically, as shown by measurement A in FIG. 3. The forward end 116 is about 1.1 mm wide horizontally, as shown by measurement B in FIG. 3.

The forward cutting surfaces 106 and lower side cutting surfaces 108 meet seamlessly at the lower apexes 120. The lower side cutting surfaces 108 are about 1.25 mm long vertically, as shown by measurement C in FIG. 3. The middle cutting surfaces 110 meet the lower side cutting surfaces 108 seamlessly at the upper end 122 of the lower side cutting surfaces 108. The middle cutting surfaces 110 are about 0.4 mm long vertically, as shown by measurement D in FIG. 3. The middle cutting surfaces 110 meet the upper side cutting surfaces 112 seamlessly at the upper apexes 124. The upper side cutting surfaces 112 are about 0.15 mm deep horizontally, as shown by measurement E in FIG. 3. The lower side cutting surfaces 108 are also about 0.15 mm deep horizontally.

The straight surgical blade 100 has three horizontal guide lines that are marked, etched, or are otherwise visible on the surface of the blade. The first guide line 126 is positioned about 0.25 mm above the forward point 118. The second guide line 128 is positioned about 0.75 mm above the forward point 118. The third guide line 130 is positioned about 1.0 mm above the forward point 118. The guide lines may be broken lines, as shown in FIG. 3 or may be unbroken. The shaft and handle of the straight surgical blade 100 can be straight or bent.

### V-Shaped Surgical Blade

FIGS. 4a and 4b show a preferred embodiment of a V-shaped surgical blade 200 adapted for use in vitreo-retinal surgery, in accordance with the principles of the present invention. The V-shaped surgical blade 200 has two opposed cutting surfaces 202 that mirror each other and substantially surround a center portion 204. The two opposed cutting surfaces 202 are comprised of two forward cutting surfaces 206, two lower side cutting surfaces 208, two middle cutting surfaces 210, and two upper side cutting surfaces 212. The two forward cutting surfaces 206 meet at the forward point 214 of the center portion 204.

Together, the two forward cutting surfaces 206 form a generally triangular-shaped forward end 216 of the V-shaped surgical blade 200, having a forward point 218 and two lower apexes 220. At the forward point 218, the cutting surface is about 0.25 mm deep vertically, as shown by measurement A in FIG. 4a. The forward end 216 of the V-shaped surgical blade 200 is about 0.5-1.6 mm wide horizontally.

The forward cutting surfaces 206 and lower side cutting surfaces 208 meet seamlessly at the lower apexes 220. The lower side cutting surfaces 208 are about 1.25 mm long vertically, as shown by measurement B in FIG. 4a. The middle cutting surfaces 210 meet the lower side cutting surfaces 208 seamlessly at the upper end 222 of the lower side cutting surfaces 208. The middle cutting surfaces 210 are about 0.4 mm long vertically, as shown by measurement C in FIG. 4a. The middle cutting surfaces 210 meet the upper side cutting surfaces 212 seamlessly at the upper apexes 224. As with the straight surgical blade 100, the lower side cutting surfaces 208 and upper side cutting surfaces 212 of the V-shaped surgical blade 200 are about 0.15 mm deep horizontally.

The V-shaped surgical blade 200 has three horizontal guide lines that are marked, etched, or are otherwise visible on the surface of the blade. The first guide line 226 is positioned about 0.25 mm above the forward point 218. The second guide line 228 is positioned about 0.75 mm above the forward point 218. The third guide line 230 is positioned about 1.0 mm above the forward point 218. The guide lines may be broken lines, as shown in FIG. 4a or may be unbroken.

Unlike the straight surgical blade 100 the V-shaped surgical blade 200 is bent in the middle along medial line 232. As shown by measurement D in FIGS. 4a and 4b, the horizontal distance from the outer edge of one of the upper side cutting surfaces 212 to the medial line 232 is about 0.25-0.9 mm. As shown by measurement E in FIG. 4b, the front-to-back distance from the outer edges of the upper side cutting surfaces 212 to the medial line 232 is about 0.35 mm. The shaft and handle of the V-shaped surgical blade 200 can be straight or bent.

### W-Shaped Surgical Blade

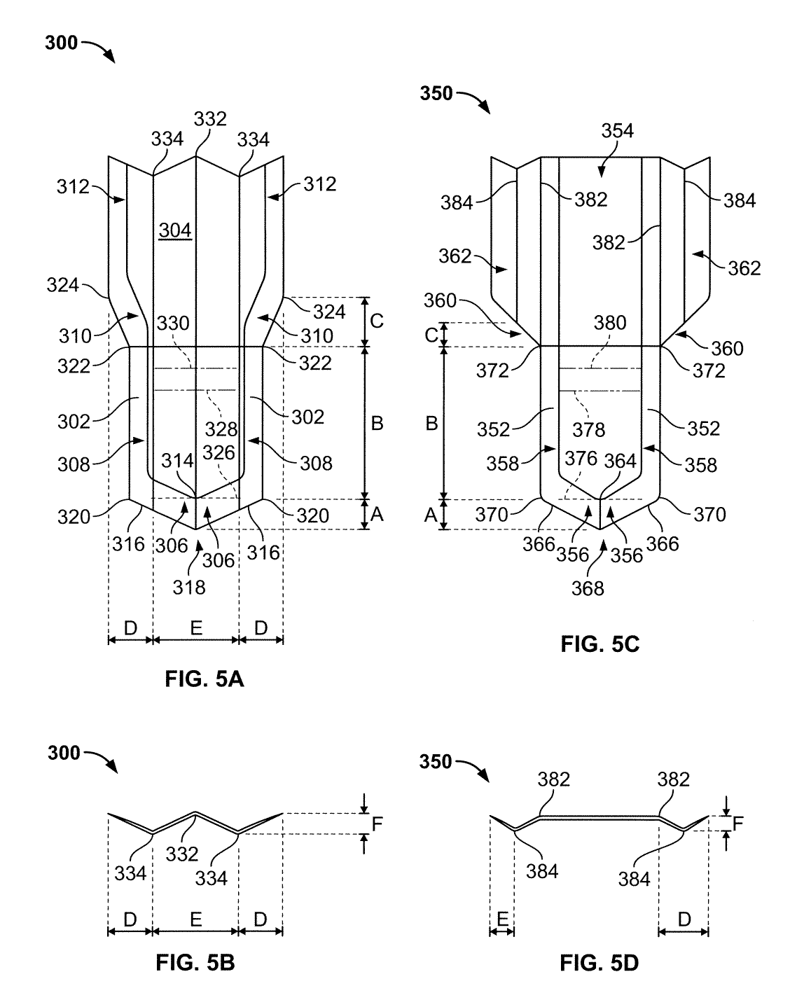

FIGS. 5a and 5b show a preferred embodiment of a W-shaped surgical blade 300 adapted for use in vitreo-retinal surgery, in accordance with the principles of the present invention. The W-shaped surgical blade 300 has two opposed cutting surfaces 302 that mirror each other and substantially surround a center portion 304. The two opposed cutting surfaces 302 are comprised of two forward cutting surfaces 306, two lower side cutting surfaces 308, two middle cutting surfaces 310, and two upper side cutting surfaces 312. The two forward cutting surfaces 306 meet at the forward point 314 of the center portion 34.

Together, the two forward cutting surfaces 306 form a generally triangular-shaped forward end 316 of the W-shaped surgical blade 300, having a forward point 318 and two lower apexes 320. At the forward point 318, the cutting surface is about 0.25 mm deep vertically, as shown by measurement A in FIG. 5a.

The forward cutting surfaces 306 and lower side cutting surfaces 308 meet seamlessly at the lower apexes 320. The lower side cutting surfaces 308 are about 1.25 mm long vertically, as shown by measurement B in FIG. 5a. The middle cutting surfaces 310 meet the lower side cutting surfaces 308 seamlessly at the upper end 322 of the lower side cutting surfaces 308. The middle cutting surfaces 310 are about 0.4 mm long vertically, as shown by measurement C in FIG. 5a. The middle cutting surfaces 310 meet the upper side cutting surfaces 312 seamlessly at the upper apexes 324. As with the straight surgical blade 100 and V-shaped surgical blade 200, the lower side cutting surfaces 308 and upper side cutting surfaces 312 of the W-shaped surgical blade 300 are about 0.15 mm deep horizontally.

The W-shaped surgical blade 300 has three horizontal guide lines that are marked, etched, or are otherwise visible on the surface of the blade. The first guide line 326 is positioned about 0.25 mm above the forward point 318. The second guide line 328 is positioned about 0.75 mm above the forward point 318. The third guide line 330 is positioned about 1.0 mm above the forward point 318. The guide lines may be broken lines, as shown in FIG. 5a or may be unbroken.

The W-shaped surgical blade 300 is bent in three places, along medial line 332 and offset lines 334. As with the V-shaped surgical blade 200, the medial line 332 bisects the W-shaped surgical blade 300. As shown by measurement D in FIGS. 5a and 5b, the horizontal distance from the outer edge of one of the upper side cutting surfaces 312 to the nearest of offset lines 334 is about 0.25 mm. As shown by measurement E in FIGS. 5a and 5b, the horizontal distance between the offset lines 334 is about 0.5 mm. As shown by measurement F in FIG. 5b, the front-to-back distance from the outer edges of the upper side cutting surfaces 312 to the offset lines 334 is about 0.15 mm. The shaft and handle of the W-shaped surgical blade 300 can be straight or bent.

### “Extended W” Shaped Surgical Blade

FIGS. 5c and 5d show a preferred embodiment of an “extended W” shaped surgical blade 350 adapted for use in vitreo-retinal surgery, in accordance with the principles of the present invention. The “extended W” shaped surgical blade 350 has two opposed cutting surfaces 352 that mirror each other and substantially surround a center portion 354. The two opposed cutting surfaces 352 are comprised of two forward cutting surfaces 356, two lower side cutting surfaces 358, two middle cutting surfaces 360, and two upper side cutting surfaces 362. The two forward cutting surfaces 356 meet at the forward point 364 of the center portion 354.

Together, the two forward cutting surfaces 356 form a generally triangular-shaped forward end 366 of the “extended W” shaped surgical blade 350, having a forward point 368 and two lower apexes 370. At the forward point 368, the cutting surface is about 0.25 mm deep vertically, as shown by measurement A in FIG. 5c.

The forward cutting surfaces 356 and lower side cutting surfaces 358 meet seamlessly at the lower apexes 370. The lower side cutting surfaces 358 are about 1.25 mm long vertically, as shown by measurement B in FIG. 5c. The middle cutting surfaces 360 meet the lower side cutting surfaces 358 at the upper end 372 of the lower side cutting surfaces 358. In the preferred embodiment of FIGS. 5c and 5d, the middle cutting surfaces 360 extend downward away from the lower side cutting surfaces 358. The middle cutting surfaces 360 are about 0.2 mm long vertically, as shown by measurement C in FIG. 5c.

The “extended W” shaped surgical blade 350 has three horizontal guide lines that are marked, etched, or are otherwise visible on the surface of the blade. The first guide line 376 is positioned about 0.25 mm above the forward point 368. The second guide line 378 is positioned about 0.75 mm above the forward point 368. The third guide line 380 is positioned about 1.0 mm above the forward point 368. The guide lines may be broken lines, as shown in FIG. 5c or may be unbroken.

The “extended W” shaped surgical blade 350 is bent in four places, along inner lines 382 and outer lines 384. As shown by measurement D in FIGS. 5c and 5d, the horizontal distance from the outer edge of one of the upper side cutting surfaces 362 to the nearest of the inner lines 382 is about 0.6 mm. As shown by measurement E in FIGS. 5c and 5d, the horizontal distance from the outer edge of one of the upper side cutting surfaces 362 to the nearest of the outer lines 384 is about 0.15-0.3 mm. As shown by measurement F in FIG. 5b, the front-to-back distance from the outer edges of the upper side cutting surfaces 362 to the outer lines 384 is about 0.15 mm. The shaft and handle of the “extended W” shaped surgical blade 350 can be straight or bent.

### Shelf-Sealing Incision

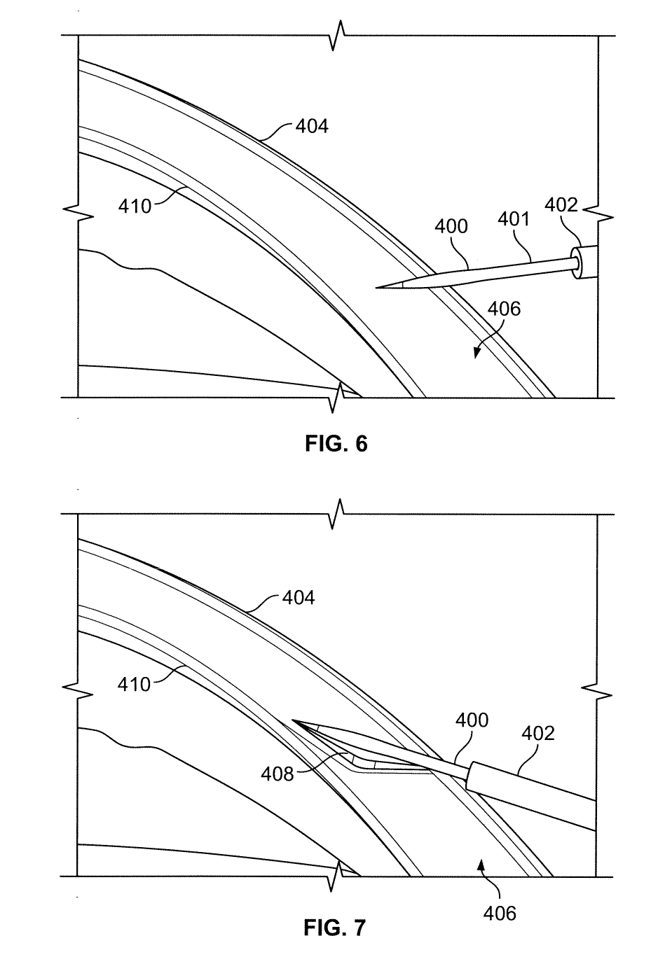

FIGS. 6 through 8 show a preferred method of creating a self-sealing incision during vitreo-retinal surgery, in accordance with the principles of the present invention.

As shown in FIG. 6, the surgeon uses a surgical blade 400, which can be any of the straight surgical blade 100, V-shaped surgical blade 200, W-shaped surgical blade 300, “extended W” shaped surgical blade 350, or any other surgical blade adapted for cutting through scleral tissue. The surgical blade has a shaft 401 connected to the cutting surface and surrounded at least partly by the sleeve of the trocar system. Holding the handle 402, the surgeon first directs the surgical blade 400 substantially perpendicular to the scleral surface 404, creating a wound about 1.0 mm wide to a depth of about 0.25 mm in the sclera 406. This 0.25 mm depth is marked on the surface of the straight surgical blade 100 as the first guide line 126. The 0.25 mm depth is marked on the surface of the V-shaped surgical blade 200 as the first guide line 226. The 0.25 mm depth is marked on the surface of the W-shaped surgical blade 300 as the first guide line 326. The 0.25 mm depth is marked on the surface of the “extended W” shaped surgical blade 350 as the first guide line 376.

As shown in FIG. 7, the surgeon next redirects the blade 400 away from a position substantially orthogonal to the scleral surface to follow the general curvature of the sclera 406. The surgeon then advances the blade 400, creating an approximately 0.75 to 1.0 mm tunnel 408. The 0.75 mm and 1.0 mm measurements are marked on the surface of the straight surgical blade 100 as the second guide line 128 and third guide line 130, respectively. The 0.75 nm and 1.0 mm measurements are marked on the surface of the V-shaped surgical blade 200 as the second guide line 228 and third guide line 230, respectively. The 0.75 mm and 1.0 mm measurements are marked on the surface of the W-shaped surgical blade 300 as the second guide line 328 and third guide line 330, respectively. The 0.75 mm and 1.0 mm measurements are marked on the surface of the “extended W′” shaped surgical blade 350 as the second guide line 378 and third guide line 380, respectively.

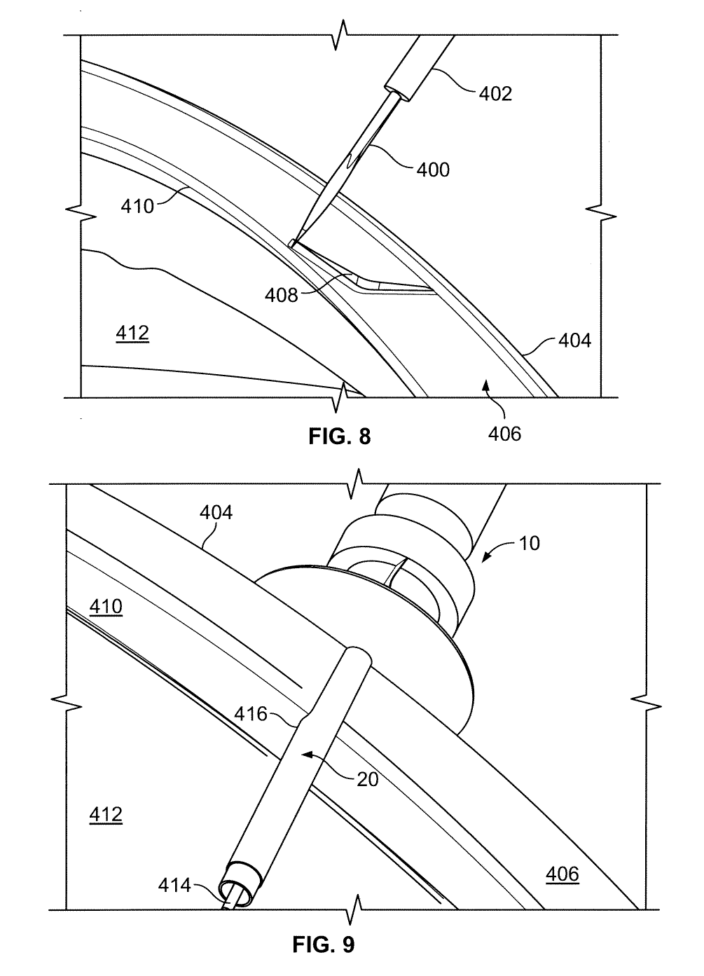

As shown in FIG. 8, the surgeon then pivots the blade back to a position substantially orthogonal to the scleral surface and advances the blade 400 to create a full-thickness sclerotomy, piercing the bottom 410 of the sclera 406 and entering the interior 412 of the eye. The sleeve 20 of the trocar system 10 is pushed through the wound along with the blade 400, and is securely in place spanning the wound after the blade 400 pierces the bottom 410 of the sclera 406. The sleeve can be pushed through the wound because, although the sleeve is easily collapsible latitudinally, it is relatively rigid longitudinally. After the incision is complete, the surgical blade can then be withdrawn from the sleeve.

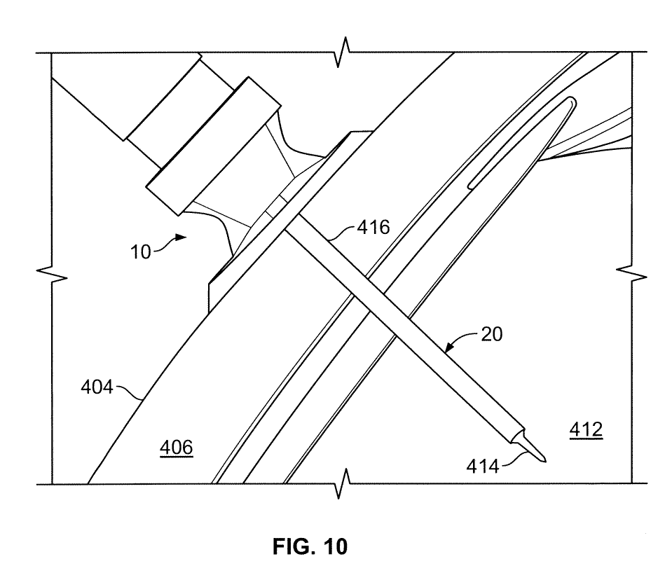

FIGS. 9 and 10 show, respectively, a perspective cross-sectional view and a side cross-sectional view of a preferred embodiment of the trocar system 10, in accordance with the principles of the present invention. The trocar system 10 is shown inserted into the interior 412 of the eye along with a surgical instrument 414 inserted into the trocar system. Surgical instrument 414 can be any 20-gauge or smaller instrument adapted for use in vitreo-retinal surgery. In embodiments for which the trocar system is adapted for use in cataract surgery, the trocar system can be used with any standard-size instrument adapted for use in cataract surgery. As shown in FIGS. 9 and 10, the shape of the sleeve 20 conforms to the shape of the surgical instrument 414. The surgical wound 416 conforms to the shape of the sleeve 20, such that the surgical wound 416 forms a relatively straight path from the scleral surface 404, through the sclera 406, to the bottom 410 of the sclera 406.

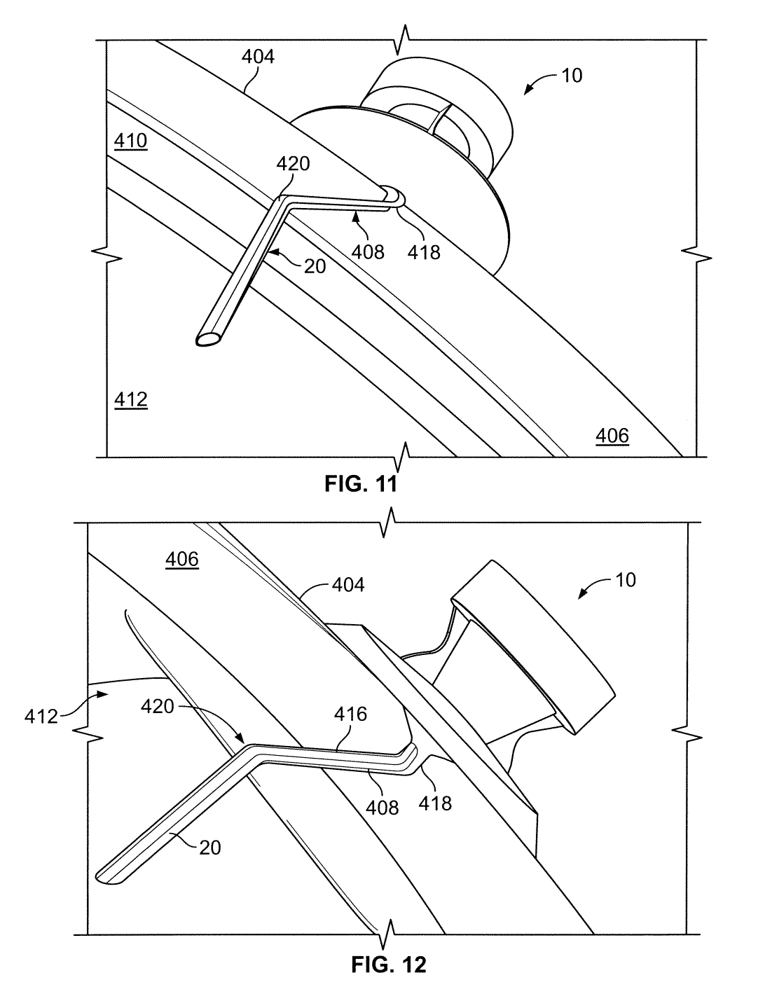

FIGS. 11 and 12 show, respectively, a perspective cross-sectional view and a side cross-sectional view of a preferred embodiment of the trocar system 10, in accordance with the principles of the present invention. The trocar system 10 is shown inserted into the interior 412 of the eye but without a surgical instrument inserted into the trocar system. The shape of the sleeve 20 conforms to the shape of the surgical wound 416 as cut by the surgeon when the surgical instrument has been removed from the trocar system. As shown in FIGS. 11 and 12, the surgical wound 416 has a first part 418 that travels substantially perpendicular to the scleral surface 404 to a depth of about 0.25 mm in the sclera 406, the tunnel 408, and a third part 420 that travels substantially perpendicular to the bottom 410 of the sclera 406, piercing the bottom 410 and entering the interior 412 of the eye.

The present invention has been described above in terms of presently preferred embodiments so that an understanding of the present invention can be conveyed. However, there are other embodiments not specifically described herein for which the present invention is applicable. Therefore, the present invention should not to be seen as limited to the forms shown, which is to be considered illustrative rather than restrictive.

I claim:

1. A trocar system for use in surgery on an eye, the trocar system comprising.
a surgical blade comprising:
a cutting surface, and
a shaft connected to the cutting surface,
wherein the surgical blade is sized to create an approximately 20-gauge to approximately 25-gauge incision in the surface of the eye;
a generally tubular sleeve having a proximal end and a distal end, wherein:
the sleeve is configured to surround at least a portion of the shaft of the surgical blade, and
the sleeve is configured to substantially mold to the shape of the incision when the surgical blade is withdrawn from the sleeve; and
an outer segment connected to the proximal end of the sleeve, the outer segment comprising:
a generally funnel-shaped external guide piece, and
a stability platform connected to the external guide piece,
wherein the stability platform is generally shaped to mate to the surface of the eye.

2. The trocar system of claim 1, wherein:
the surgical blade further comprises a center portion aligned along a longitudinal axis of the surgical blade;
the cutting surface further comprises:
a forward cutting surface having a forward point, a first end, and a second end,
a first side cutting surface connected to the first end of the forward cutting surface, and
a second side cutting surface connected to the second end of the forward cutting surface;
the center portion comprises:
a first guide marker spaced approximately 0.25 mm from the forward point of the forward cutting surface,
a second guide marker spaced approximately 0.75 mm from the forward point of the forward cutting surface, and
a third guide marker spaced approximately 1.0 mm from the forward point of the forward cutting surface; and
the cutting surface substantially surrounds the center portion.

3. The trocar system of claim 2, wherein the forward cutting surface is approximately 0.25 mm deep at the forward point measured in a direction parallel to the longitudinal axis of the surgical blade.

4. The trocar system of claim 2, wherein the first side cutting surface and the second side cutting surface are aligned substantially parallel to the longitudinal axis of the surgical blade.

5. The trocar system of claim 4, wherein:
the first side cutting surface is approximately 0.15 mm deep, measured in a direction orthogonal to the longitudinal axis of the surgical blade; and
the second side cutting surface is approximately 0.15 mm deep, measured in a direction orthogonal to the longitudinal axis of the surgical blade.

6. The trocar system of claim 1, wherein the surgical blade is bent along a longitudinal axis of the surgical blade, such that the surgical blade has a V-shaped cross-section.

7. The trocar system of claim 1, wherein the surgical blade is bent along a longitudinal axis of the surgical blade and along two offset lines that are parallel to the longitudinal axis, such that the surgical blade has a W-shaped cross-section.

8. The trocar system of claim 7, wherein the two offset lines are spaced approximately 0.5 mm apart.

9. The trocar system of claim 1, wherein the surgical blade is bent along a plurality of offset lines that are parallel to a longitudinal axis of the surgical blade.

10. The trocar system of claim 1, wherein the sleeve comprises a material selected from the group consisting of plastic polymers and metal mesh.

11. The trocar system of claim 10 wherein the material is a fenestrated plastic polymer.

12. The trocar system of claim 10, wherein the material is a plastic polymer that is relatively rigid longitudinally and relatively collapsible latitudinally.

13. The trocar system of claim 1, wherein:
the proximal end of the sleeve defines an approximately 20-gauge opening; and
the distal end of the sleeve defines an approximately 20-gauge opening.

14. The trocar system of claim 1, wherein the outer segment comprises a material that glows in the dark.

15. The trocar system of claim 1, wherein the outer segment defines an opening that is greater than approximately 18 gauge.

16. A surgical blade for use in eye surgery, the surgical blade comprising:
a center portion aligned along a longitudinal axis of the surgical blade; and
a cutting surface that substantially surrounds the center portion, the cutting surface comprising:
a forward cutting surface having a forward point, a first end, and a second end,
a first side cutting surface connected to the first end of the forward cutting surface, and
a second side cutting surface connected to the second end of the forward cutting surface;
wherein the center portion comprises:
a first guide marker spaced approximately 0.25 mm from the forward point of the forward cutting surface,
a second guide marker spaced approximately 0.75 mm from the forward point of the forward cutting surface, and
a third guide marker spaced approximately 1.0 mm from the forward point of the forward cutting surface.

17. The surgical blade of claim 16, wherein the forward cutting surface is approximately 0.25 mm deep at the forward point, measured in a direction parallel to the longitudinal axis of the surgical blade.

18. The surgical blade of claim 16, wherein the first side cutting surface and the second side cutting surface are aligned substantially parallel to the longitudinal axis of the surgical blade.

19. The surgical blade of claim 18, wherein the forward cutting surface is approximately 1.1 mm wide, measured in a direction orthogonal to the longitudinal axis of the surgical blade.

20. The surgical blade of claim 18, wherein:
the first side cutting surface is approximately 0.15 mm deep, measured in a direction orthogonal to the longitudinal axis of the surgical blade; and
the second side cutting surface is approximately 0.15 mm deep, measured in a direction orthogonal to the longitudinal axis of the surgical blade.

21. The surgical blade of claim 16, wherein the surgical blade is bent along the longitudinal axis, such that the surgical blade has a V-shaped cross-section.

22. The surgical blade of claim 16, wherein the surgical blade is bent along the longitudinal axis and along two offset lines that are parallel to the longitudinal axis, such that the surgical blade has a W-shaped cross-section.

23. The surgical blade of claim 22, wherein the two offset lines are spaced approximately 0.5 mm apart.

24. The surgical blade of claim 16, wherein the surgical blade is bent along a plurality of offset lines that are parallel to the longitudinal axis.

25. A method for using a surgical blade to create a self-sealing incision in an eye sclera, the eye sclera having an exterior surface and an interior surface, wherein the surgical blade comprises a cutting surface and a shaft connected to the cutting surface, and wherein at least a portion of the shaft of the surgical blade is placed within a generally tubular sleeve, the method comprising the steps of:
advancing the surgical blade and sleeve substantially orthogonally to the exterior surface of the sclera to a depth of approximately 0.25 mm in the sclera;
pivoting the surgical blade and sleeve away from a position substantially orthogonal to the exterior surface of the sclera;
advancing the surgical blade and sleeve within the sclera to create a tunnel having a length of approximately 0.75 mm to approximately 1.0 mm;
pivoting the surgical blade and sleeve to a position substantially orthogonal to the exterior surface of the sclera;
advancing the surgical blade and sleeve substantially orthogonally to the exterior surface of the sclera to pierce the interior surface of the sclera; and
withdrawing the surgical blade from the sleeve;
wherein the sleeve is configured to substantially mold to the shape of the incision when the surgical blade is withdrawn from the sleeve.
